(19) United States
(12) Patent Application Publication
(10) Pub. No.: US 2023/0070042 A1
(43) Pub. Date: Mar. 9, 2023

(54) BYPASS SYRINGE

(71) Applicant: Biolife, L.L.C., Sarasota, FL (US)

(72) Inventors: Talmadge Kelly Keene, Ruskin, FL (US); Clayton Keene, Riverview, FL (US)

(21) Appl. No.: 17/903,768

(22) Filed: Sep. 6, 2022

Related U.S. Application Data
(60) Provisional application No. 63/240,413, filed on Sep. 3, 2021.

(51) Int. Cl. A61B 17/135

(52) U.S. Cl. CPC A61B 17/135; A61B 2017/12004

(57) ABSTRACT

The bypass syringe is used in combination with an expandable or inflatable bladder or balloon that is circumferentially wrapped around a patient designed to reduce bleeding from a skin insertion wound site and a vascular wound site after a vascular intervention or surgical procedure is performed. The bypass syringe is designed to pre-inflate the balloon, allow the balloon to relax based on how tightly the band is wrapped around the patient, and then be inflated with a volumetric amount of fluid. The bypass syringe's pre-inflation step will reduce complications caused by variabilities in band securement or tightness. A pre-inflation step combined with a venting of excess air step or controlled pressure inflation steps will absorb/take up/consume excess space caused by variability in band securement and help to reduce complication caused by securement variability.

### RELATED APPLICATION

The present application claims the benefit of U.S. Provisional Patent Application No. 63/240,413, filed on Sep. 3, 2021, the entire disclosure of which is incorporated by reference herein.

### FIELD OF THE INVENTION

The present invention relates in general to hemostasis, methods of achieving hemostasis, devices used in such methods, and, in particular, to hemostasis of a blood vessel immediately after performing a vascular catheterization procedure.

### BACKGROUND OF THE INVENTION

Many medical procedures that once required extensive invasive surgery are performed today less invasively by inserting surgical or diagnostic devices through arteries or veins (i.e., vascular procedures). These procedures are much safer and require significantly less recovery time than prior procedures. To prevent clots from forming in the vessels during and after the procedure, the patient may require anticoagulation medications, which often result in excessive bleeding. To stop bleeding after vascular procedures, direct pressure is typically applied to the wound. This pressure must be held over both the entry point wound in the skin and the wound that was created in the vessel.

Such direct pressure can be applied manually by a clinician, but in many instances the pressure is needed for an extended period of time to decrease or stop the bleeding, and there are issues regarding consistency of pressure application between clinicians. To save the clinicians time and to improve on consistency, many compression devices have been developed. Some of such devices employ an inflatable bladder or balloon with a circumferential band to create the compression needed to stop the bleeding. First the band is wrapped around the limb or other body part, and then the bladder is inflated to create compression pressure over the wound site. As stated previously, the compression must cover both the wound in the skin as well as the wound in the vessel beneath the skin.

Recent testing has revealed that the variabilities in band securement or tightness around the limb has an impact on the compression applied as the balloon is inflated. A tighter band, on the same patient, with the same volume of air will create more compression than will a more loosely applied band. Further, multiple variables can make training for band attachment a difficult and inconsistent process. These factors make it difficult to create and apply a standard volumetric inflation hemostasis protocol. A solution to this issue has been found in partially inflating the balloon prior to the volumetric inflation. The degree to which the band is inflated prior to volumetric inflation will be dependent on how loosely or tightly the band is applied, such that looser bands will inflate more than tighter bands prior to volumetric inflation. This lessens the impact that band tightness has on compression force with requiring a lengthy and expensive training procedure for the clinician.

The present invention addresses a method of creating consistent pressure on both wounds from patient to patient that is not possible with any current compression band systems on the market. This is accomplished by creating a mechanism that pre-inflates the balloon on the band with a near atmospheric pressure air to compensate for the variability in band securement tightness. A tightly applied band would have less air in the balloon than would a loosely applied band. The larger volume of air would take up the additional space created by the loosely applied band. Afterwards, a measured volume of air could be injected to create more uniform compression over the site.

The currently marketed bands typically consist of a syringe for inflating; a check valve to hold air in the balloon once inflated; a tube connecting the check valve to the balloon; a circumferential band, most often employing Velcro® as an attachment method to hold the band in place around the limb of the patient; and an inflatable balloon.

The compression must stop bleeding at the surface as well as bleeding at the artery. Bleeding from the artery that does not exit the body will create a hematoma.

### SUMMARY OF THE INVENTION

This invention relates generally to using a syringe to deliver air into a balloon or inflatable bladder to inflate the balloon with near atmospheric air pressure, before delivering a measured volume of air. The balloon or bladder is held on to the patient with a circumferential wrap. Typical syringes for radial hemostasis are 25 ml syringes. The transition from pressure control to volumetric control could be accomplished with a mechanical bypass system to inflate a bladder or balloon to improve hemostasis after a surgical procedure (such as a vascular access procedure), or a syringe with a pressure relief valve to pressurize a balloon to a given pressure prior to delivering a measured volume of air. A similar system could be created as part of the check valve apparatus, connective tubing, or the balloon for commercially available band.

Briefly, the present invention is directed, in an embodiment, to a device for creating improved hemostasis, wherein the device comprises a syringe, with continuous channel(s) or opening(s) or slit(s) or intermittent openings (holes) connected with external pieces. The holes or slits are positioned such that they allow air to bypass the plunger seal as the plunger is being depressed. Above the top hole or slit and below the bottom hole or slit, the syringe will deliver a volumetric amount of air into the balloon. When the syringe plunger is between the top and bottom holes or in the area of a slit, the syringe will allow air from the balloon to push back into the syringe, bypass the plunger, and escape from the system. The holes or slits may be located along a single point on the external radius of the cross section of the barrel, or they may be located at multiple points on the external radius of the cross section of the barrel (a continuous opening would manifest as a slit running diagonally along the barrel of the syringe, such that the slit traverses it both laterally and circumferentially). Additionally, a telescoping apparatus may be placed at the base of the syringe which can be expanded to seal the holes running up the syringe. This will allow for a balloon to be slightly inflated and to relax to take up excess space between the balloon and patient caused by a band that is applied too loosely.

In another embodiment, the invention is directed to a syringe with a pressure relief valve in the barrel located at a measured volume from the bottom of the syringe. When the plunger is above the pressure relief valve, the syringe would deliver air to the balloon on a pressurized basis, and below the valve, the syringe would deliver air on a volumetric basis. This would allow for a balloon to be inflated based on pressure to take up excess space between the balloon and patient caused by a band that is applied too loosely when the plunger is above the valve, and then deliver a volumetric amount of air below the valve. The pressure relief valve may be a mechanical spring valve, a simple hole with a controlled orifice, or a hole with a covering to restrict air flow. When used, the covering may be solid or contain an opening itself.

In certain embodiments, the bypass system could be part of the check valve, connecting tubing, or the balloon itself. To use any of the bypass systems contemplated herein, the inflation steps are: 1) apply the compression device to the patient's wound site; 2) slightly pressurize the balloon; 3) allow the balloon to relax to fill the excess space between the band and the patient; and then 4) fill the partially-inflated relaxed band with a measured volume of air. This procedure may require manipulation of the syringe, or the pressure relief mechanism during the inflation steps. In an alternative embodiment, a variable pressure relief valve on a band or syringe with a preset desired pressure could be used.

In another embodiment, a method of using an inflatable balloon band could be to preinflate the balloon prior to application or after the band is applied to the patient yet before the inflation step designed to apply patent hemostasis compression pressure over the radial artery. Then prior to the measured inflation step allowing the balloon to deflate to a point of relaxation based on the level of securement around the wrist, by venting the valve to the balloon. Afterwards inflating the balloon with a measured volume of liquid to apply a compression force over the radial artery to create patent hemostasis. The preinflation then relaxation step will allow the remaining air in the balloon to compensate for the variability of band securement tightness. If the band is tightly secured around the wrist the relaxation step would allow for most of the air to be released from the balloon, while with a loosely applied band the balloon would retain more air during the relaxation step, due to the space created by the loosely applied band.

A protocol using the method from the previous paragraph would be to apply the band around the patient's wrist. Inflate the balloon with 5 ml of air. Disconnect the syringe from the check valve, remove the plunger from the syringe, then reinsert the syringe barrel into the check valve allowing the balloon to relax. Then remove the syringe barrel from the check valve, reinsert the plunger into the syringe barrel, set the plunger to the desired volume of air, and inject that air into the balloon. This protocol could simulate the inflation mechanism produced by the syringe with the bypass channel. The key is the relaxation step where the balloon remains inflated with air near atmospheric pressure, and that near atmospheric volume of air will account for some of the variability in band securement tightness.

In another embodiment, a syringe plunger may be modified using a spring having a selected spring constant to indicate the force applied to the plunger. In conjunction with the plunger seal diameter, the device will deliver fluid of proportional pressure into an inflatable band.

### BRIEF DESCRIPTION OF THE DRAWINGS

A full and enabling disclosure of the present invention, including the best mode thereof directed to one of ordinary skill in the art, is set forth in the specification, which refers to the appended drawings, in which:

FIG. 1 illustrates a typical type of vascular access.

FIG. 2 shows the results of varied compression over access sites.

FIGS. 3-11 illustrate the operation of one embodiment of the bypass syringe.

FIGS. 12-15 illustrate an embodiment for the present invention including a pressure relief valve in the barrel of the syringe which can be a preset valve or a variable valve manipulable by the clinician.

FIGS. 16-17 illustrate an example, and use, of a pressure relief valve incorporated into a syringe plunger.

FIG. 18 is a sketch of the compressible feedback syringe plunger (without the syringe barrel).

FIG. 19 shows a test model used for the present invention.

FIG. 20 shows more of the test model of FIG. 19 with a data acquisition system connected.

FIG. 21 illustrates different syringes and setups used for testing a particular patient.

FIG. 22 is a graph showing the compression for exerted over the radial artery for a typical actual use, or a TR Band® and a StatSeal RAD Disc®.

FIG. 23 is a graph demonstrating the advantage that the syringe with an 1/16″ hole at 8 ml has over an unmodified syringe.

FIG. 24 shows the results of a study related to the present invention.

FIG. 25 shows an alternative embodiment of the present invention.

FIG. 26 shows an alternative embodiment of the present invention.

FIG. 27 is a graph illustrating the difference in of compression force created with a syringe versus a bypass syringe at varying securements of the band.

FIG. 28 illustrates the compression force (lbs) and volume (ml) of air removed from the fully inflated balloon bands in FIG. 26.

FIG. 29 is a graph that represents the force applied by the TR Band® with a StatSeal RAD at various speeds of injection.

FIG. 30 is a graph that represents the force applied by the TR Band® with a StatSeal RAD at various speeds of injection.

FIG. 31 is a graph that illustrates the correlation of internal pressure to external force applied by TR Band® with StatSeal RAD.

FIG. 32 is a graph illustrating use of a syringe with a 1/16″ hole at the 8 ml mark was used to inflate the balloon for 1 minute.

### DETAILED DESCRIPTION OF PREFERRED EMBODIMENTS

Reference now will be made in detail to the embodiments of the invention, one or more examples of which are set forth below. Each example is provided by way of explanation of the invention, not a limitation of the invention. In fact, it will be apparent to those skilled in the art that various modifications and variations can be made in the present invention without departing from the scope or spirit of the invention. For instance, features illustrated or described as part of one embodiment, can be used on another embodiment to yield a still further embodiment. The following embodiments and aspects thereof are described and illustrated in conjunction with systems, tools and methods which are meant to be exemplary and illustrative and not limiting in scope. In various embodiments one or more of the above-described problems have been reduced or eliminated while other embodiments are directed to other improvements.

Thus, it is intended that the present invention covers such modifications and variations that come within the scope of the appended claims and their equivalents. Other objects, features and aspects of the present invention are disclosed in or are obvious from the following detailed description. It is to be understood by one of ordinary skill in the art that the present discussion is a description of exemplary embodiments only, and is not intended as limiting the broader aspects of the present invention.

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

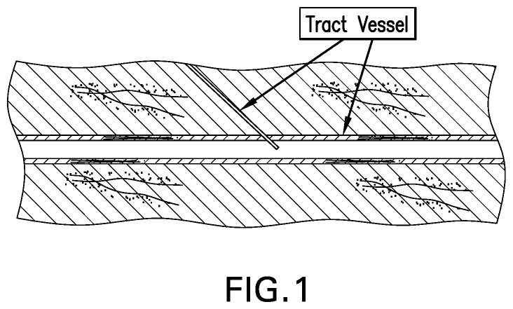

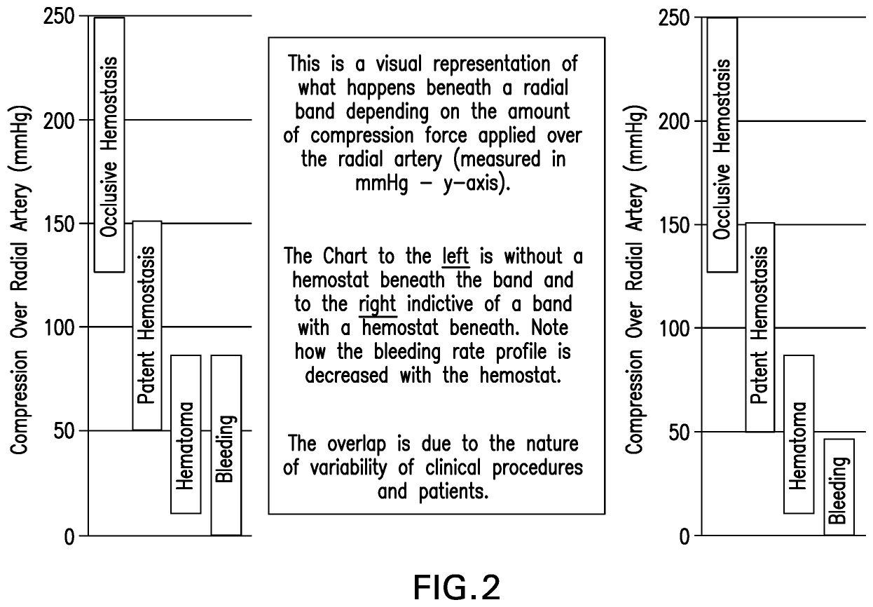

FIG. 1 shows the typical vascular access that needs compression to control hemostasis post-procedure where compression must control bleeding at the wound in the vessel and wound in the skin. FIG. 2 shows the results of varied compression over access sites. Too much pressure will occlude the artery/vessel, and too little will result in bleeding or a hematoma. Ideal compression is termed “Patent Hemostasis.” Patent Hemostasis is sufficient pressure to stop bleeding at the skin and artery/vessel, not so much as to compress the artery/vessel closed.

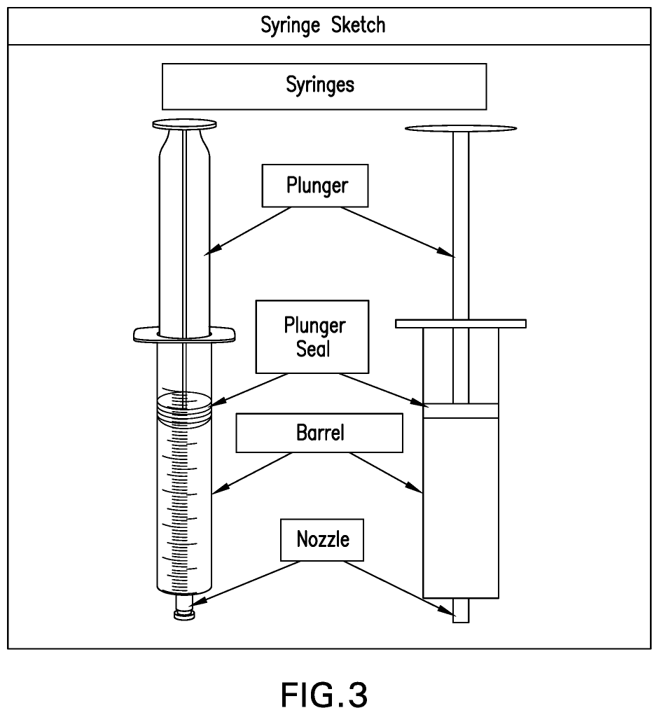

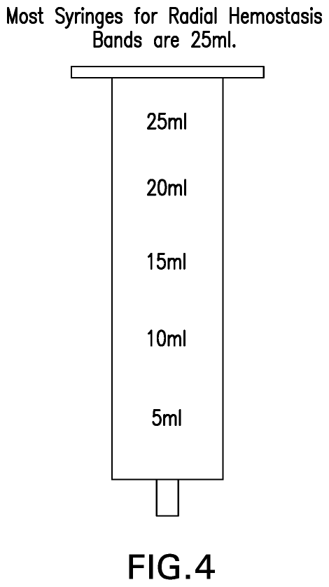

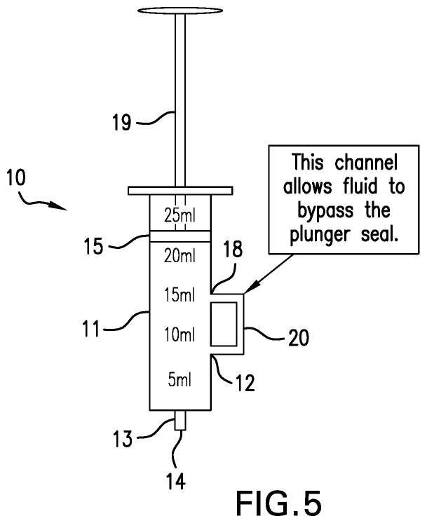

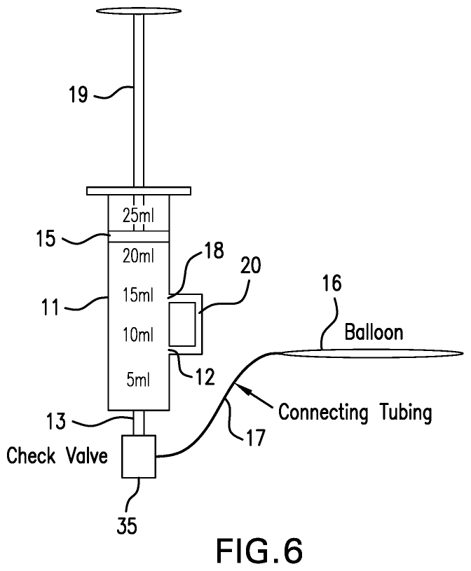

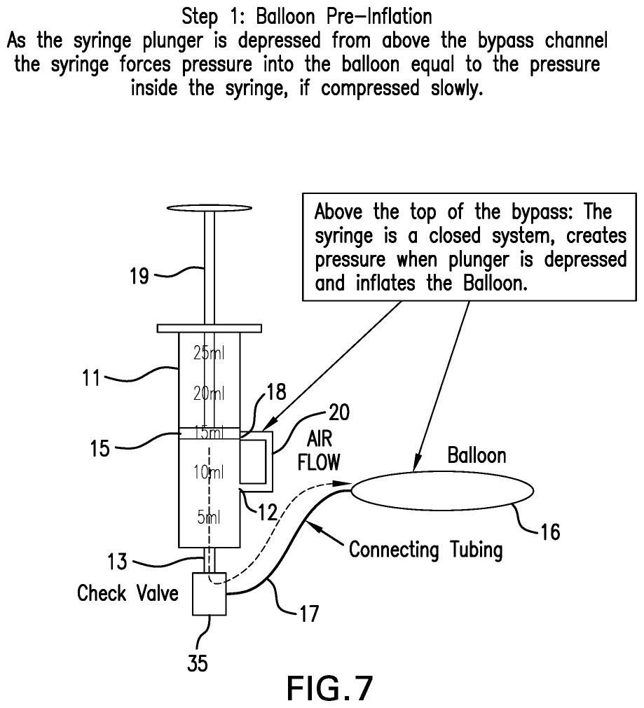

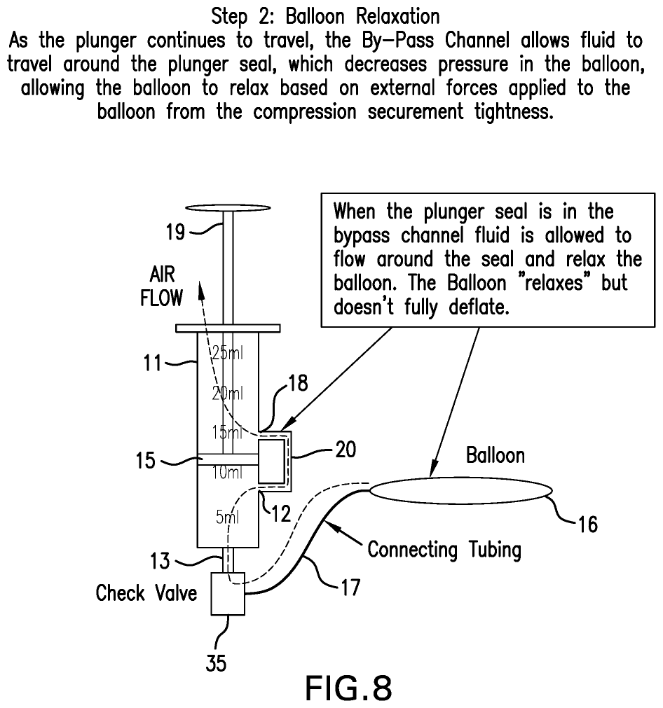

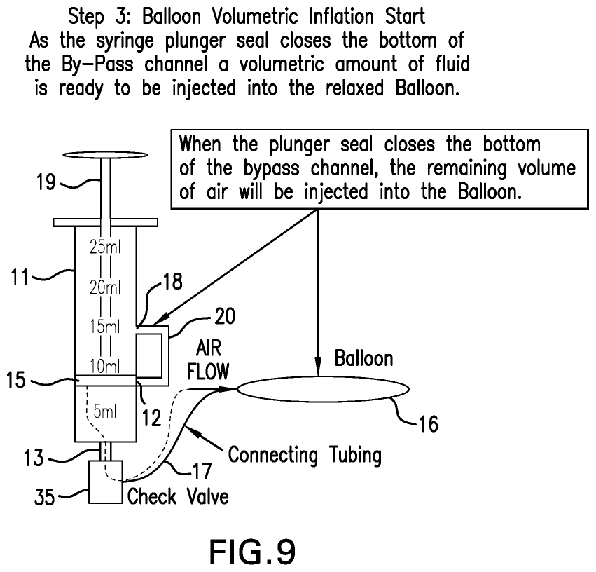

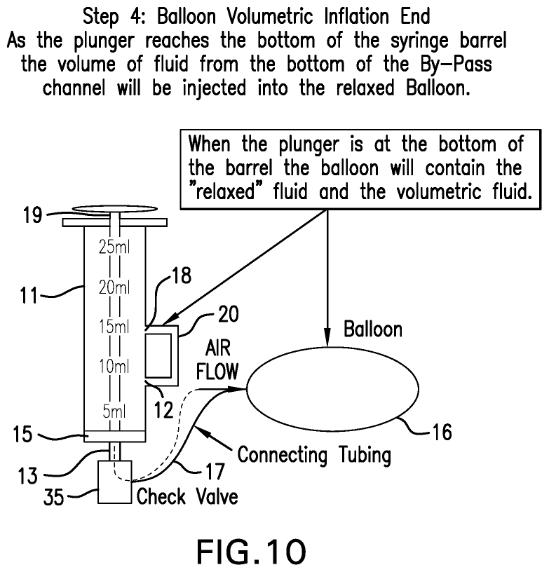

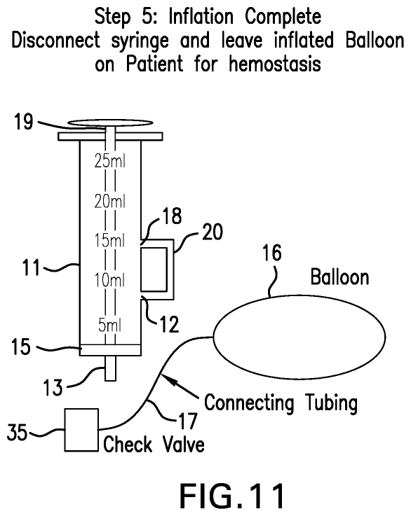

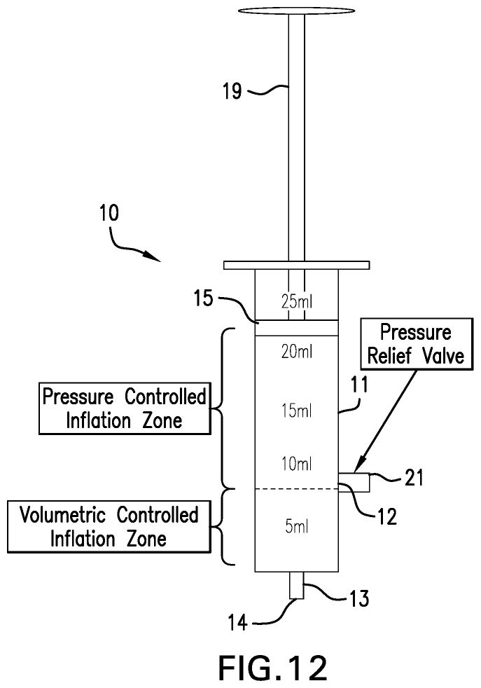

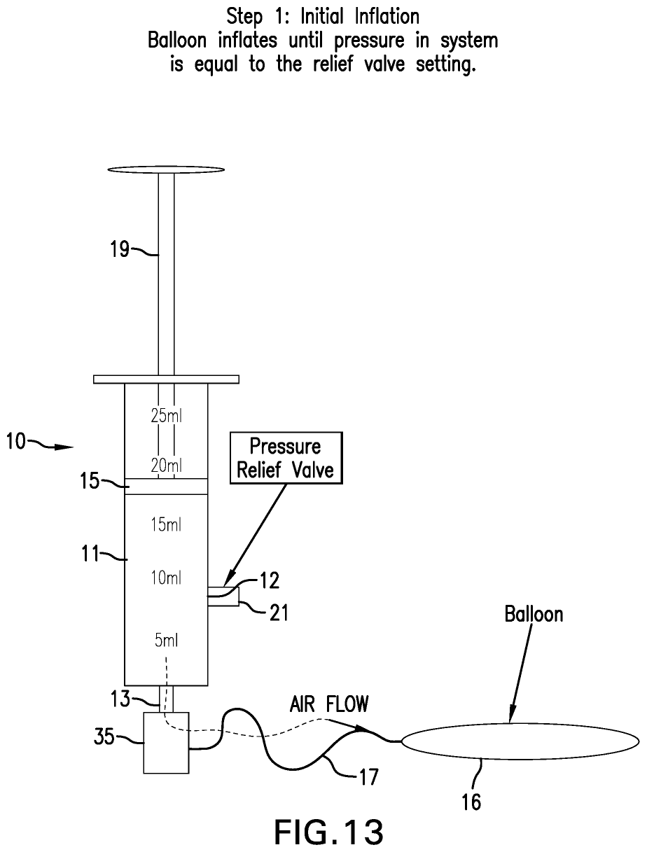

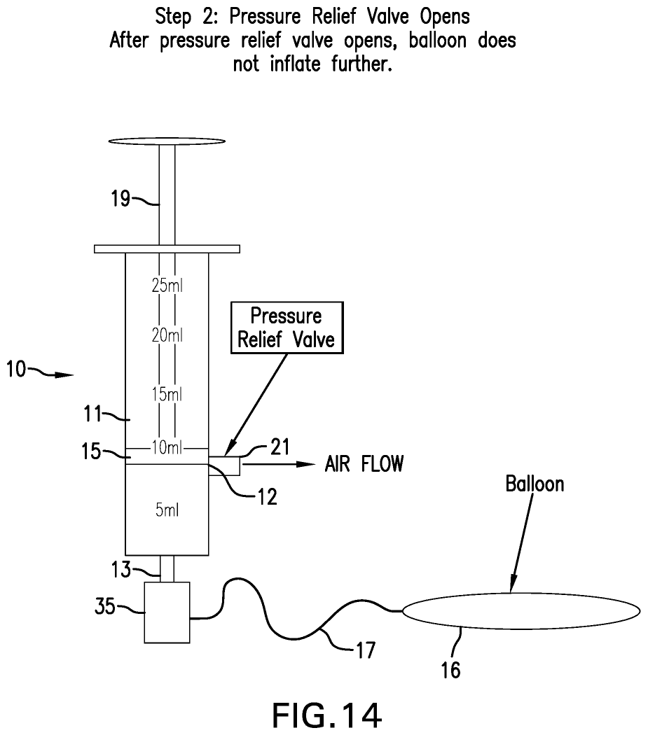

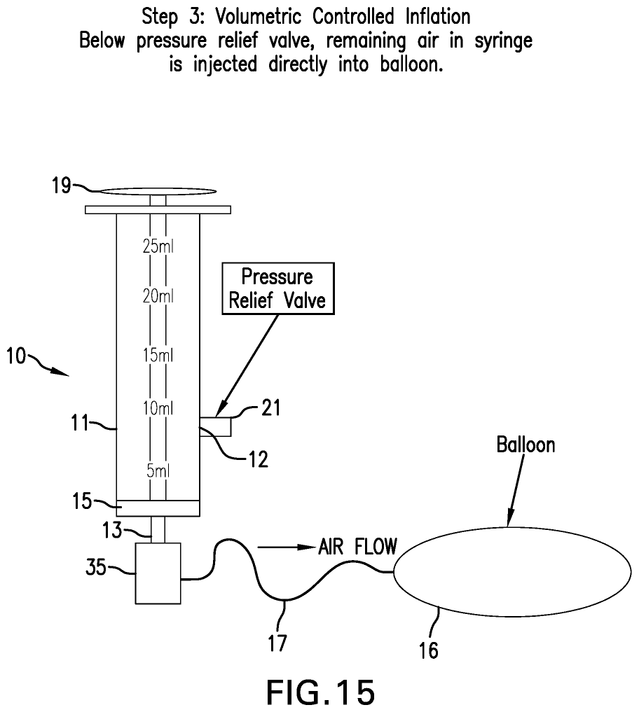

FIGS. 3-11 illustrate the operation of one embodiment of the bypass syringe 10. As shown in FIGS. 12-15, one bypass syringe 10 in accordance with the present invention is a syringe into which a small hole 12 (or multiples holes 12 and 18 as shown in FIGS. 5-11 and 25) has been created in the syringe barrel 11. The hole 12 will be similar in size to the orifice (nozzle opening 14) in the nozzle 13 to force some air into the balloon 16 and out of the hole 12 in the barrel 11 when used. The ratio of the size of the syringe holes 12 to the diameter of the nozzle opening 14 will dictate how quickly the balloon 16 is inflated. The hole 11 may be placed 10-65% from the bottom of the syringe to allow the syringe to inject all of the remaining 10-65% of the volume of the syringe into the slightly inflated balloon. In one example, the syringe may have a 1/16-inch hole in the barrel 11 at the 8 ml mark on the syringe. As the plunger 19 is depressed, before the plunger seal 15 reaches the 8 ml mark, fluid can exit the hole in the barrel 11 or the hole 14 in the nozzle 13 or tip of the syringe, slightly inflating the balloon 16 (which may be connected to the bypass syringe 10 by connecting tubing 17). A check valve 35 may be used to maintain balloon inflation when the bypass syringe 10 is not in communication with balloon 16. How much air flows into the balloon 16, through the nozzle 13, will depend on how tightly the band (not shown) is wrapped around the patient. Once the plunger seal 13 passes the 8 ml mark, air can no longer exit the hole 12 in the barrel 11 and air must exit the nozzle 13, resulting in an injection of 8 ml of air into the slightly inflated balloon 16. The 8 ml is in addition to the any air that was injected in the previous step.

As shown in FIGS. 5-11 and 25, in other embodiments, the barrel 11 may have multiple holes such as two holes 12 and 18. In another embodiments, a sliding covering, individual adhesive, or multiple coverings over each hole may be utilized to allow the clinician to determine the volume of fluid to be injected into the balloon.

In another embodiments, as shown in FIGS. 5-11, a bypass channel 20 may be located in the barrel 11 of the syringe 10 to allow air to bypass the plunger 20 as the syringe is depressed. Assuming that the average syringe has a plunger that spans approximately 10% of the lateral length of the barrel, the bottom of the bypass opening could be located 10-65% from the bottom of the syringe, and the top of the channel could be 25-80% from the bottom of the syringe. For example, in a 25 ml syringe, one end of the bypass channel opening (i.e., hole 12) could be located between 2.5-16.25 ml on the barrel of the syringe, and the other end of the bypass channel opening (i.e., hole 18) could be located between 6.25-20 ml on the barrel of the syringe. The bypass channel 20 allows air to pass around the plunger seal 15 when the plunger seal 15 is pushed to a location between the lower and higher openings of the bypass channel 20.

The bypass channel 20 may be a tube connecting two points on the syringe, or a slit that is covered by an outer sleeve, or an adhesive strip is applied over the slit. Both methods will allow a fully retracted plunger to apply pressure as it is depressed until the plunger seal passes the first side of the channel or slit. Then the syringe will vent to air until the plunger seal passes the second side of the plunger. At this point, the syringe will deliver the remaining volume into the balloon. Use of this bypass channel syringe 10 would allow the balloon to be partially inflated to overcome variability in band securement tightness, prior to a measured volumetric inflation of said balloon.

The method of pressurizing, relaxing to near atmospheric internal balloon pressure, and, ultimately, volumetric dosing to create the uniform compression may be accomplished by the steps of inflating the balloon with the syringe, removing the syringe, removing the plunger from the syringe, reinserting the syringe barrel (without plunger in place), and then allowing the balloon to relax. After relaxing, the syringe would be removed, plunger reinserted, plunger seal set to volume dosing desired, and then the measured volume would be injected into the balloon.

The bypass syringe would operate according to the following steps. For this example, the bottom bypass channel hole would be at 8 ml and the top bypass channel hole would be at 15 ml. A covering would form a channel between these two points.
1) The band would be placed on the patient.
2) The syringe plunger would be pulled back to 20-25 ml.
3) The syringe nozzle would be inserted into the check valve.
4) The syringe would be depressed:
   Pressure controlled pre-inflation step—above 15 ml, the syringe will inject all air “exiting” the syringe into the balloon. The syringe will contain pressurized air during this step.
   Balloon relaxation step—between 15 ml and 8 ml the air in the syringe and balloon will escape around the plunger seal and exit the plunger. During this time, the balloon will relax, the degree to which is dependent on how tightly the band is secured around the patient. A looser band will generate less compression on the balloon, and the balloon will retain more air during this step in the process.
   Volume-controlled inflation step—below 8 ml, the syringe will displace all air into the balloon.
5) The syringe is disconnected from the check valve and the balloon remains inflated with the relaxation air plus the 8 ml.
6) The syringe can then be used as a normal syringe below the 8 ml mark to make incremental changes to the balloon (inflation or deflation) that may be necessary in the clinical setting.

The vertical location of the bottom and top of the channel may vary. For example, a larger balloon may require more fluid in the final injection versus a smaller balloon. This could impact the position or length of the channel.

The length of the channel may also vary, but needs to be greater than the plunger seal width. Plunger seal widths may vary between syringes.

In other embodiments, multiple intermittent openings or holes in the barrel of the syringe barrel 11, connected with an external airtight piece, may be utilized. The distance between the intermittent openings may run a larger lateral distance along the barrel of the syringe than does the seal of the plunger. The intermittent openings may be located at a single or at multiple points along the outer radius of the barrel.

In other alternative embodiments, one or more continuous openings may be utilized on the barrel 11 of the syringe. The continuous openings may run a larger lateral distance along the barrel than does the plunger seal 15. The continuous openings may be located at a single point along the outer radius of the barrel, or may traverse the barrel circumferentially along the lateral length of the barrel.

Holes in the barrel 11 may be covered in a way to create volume between the barrel and the covering.

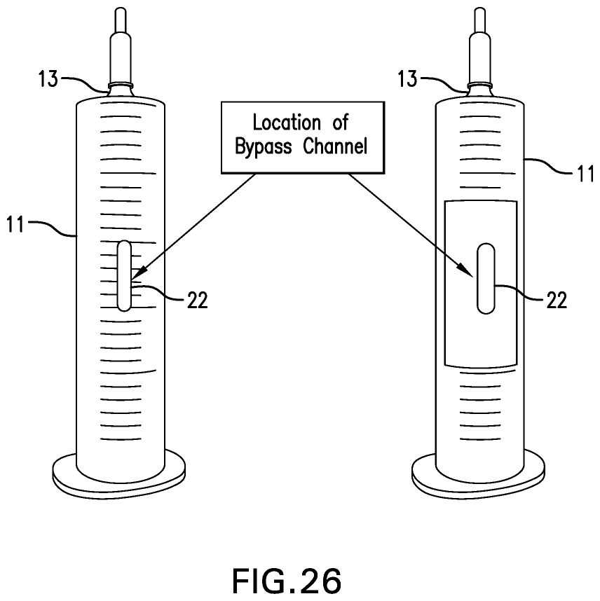

As shown in FIG. 26 a continuous opening (such as a slit) 22 could be covered with an adhesive or a simple plastic wrap around the entire barrel of the syringe.

In other embodiments, a syringe with multiple holes could employ a sliding outer sleeve to cover a portion of the holes to create a variable final volume step syringe.

In other embodiments, as shown in FIG. 12-18, a bypass syringe 10 having a pressure relief valve 21 in the barrel 11 may be utilized. The pressure relief valve 21 may be located 10-65% from the bottom of the syringe to allow the syringe to perform a final volumetric injection into the balloon. This may also allow the syringe to be used to perform small adjustments to the balloon or deflate the balloon for removal.

The pressure relief valve 21 may be set to deliver a small amount of air into the balloon, and the valve may be located to allow the syringe to inject a measured volume of air into the balloon.

The pressure relief valve 21 may be set near 100 mgHg to create patent hemostasis for certain patients. In another embodiment, a variable valve that can be set by the clinician may be used and may include a pressure point indicator.

The pressure relief valve 21 may be a variable valve for setting to a pressure by the clinician based on the patient's blood pressure. The variable relief valve may be implemented at any point along the barrel 11 of the syringe 10 or the nozzle 13 of the syringe 10, or may be incorporated into the plunger 19 of the syringe. The plunger 19 may have an open center shaft whereby air can escape through the pressure relief valve and the pressure relief valve may be controlled by a screw-tight mechanism at the end of the syringe plunger. The clinician could preset the pressure at which the valve would open, which would determine the internal pressure of the balloon, the set point of which would be determined based on the patient's blood pressure.

FIGS. 12-15 illustrate an embodiment for the present invention including a pressure relief valve 21 in the barrel 11 of the syringe 10 which can be a preset valve or a variable valve manipulable by the clinician. If a preset pressure relief valve 21 is located on the barrel 11 of the syringe 10 at 10%-30% from the bottom of the syringe 10 then the syringe 10 could be used to inflate the balloon 16 to an internal air pressure set point until the plunger seal 15 closes off the pressure relief valve 21. This preset pressure may accommodate the compression needs for most patients, but some hypertensive, or other clinical outliers may require additional pressure. The bottom portion of the syringe, under no pressure relief control, could then be used to inflate the balloon with an air pressure greater than the preset relief valve and create the increased compression over the wound site.

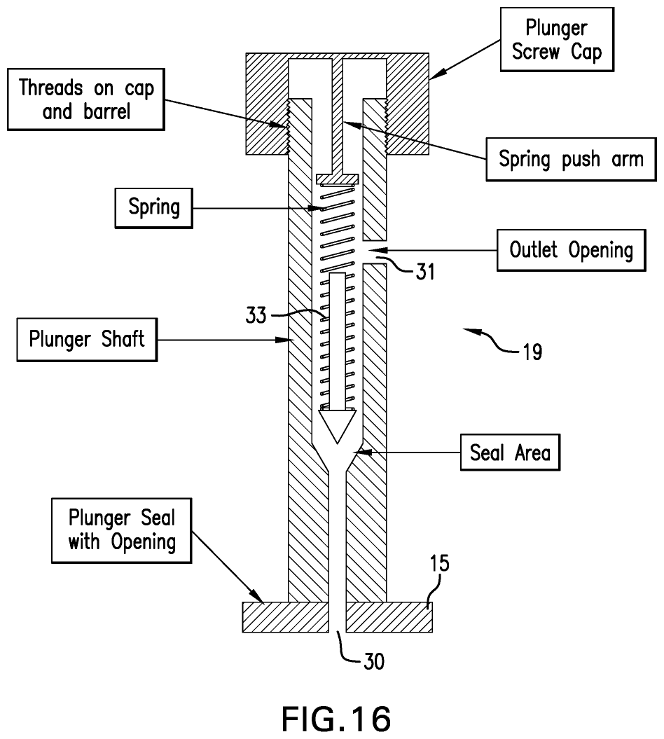

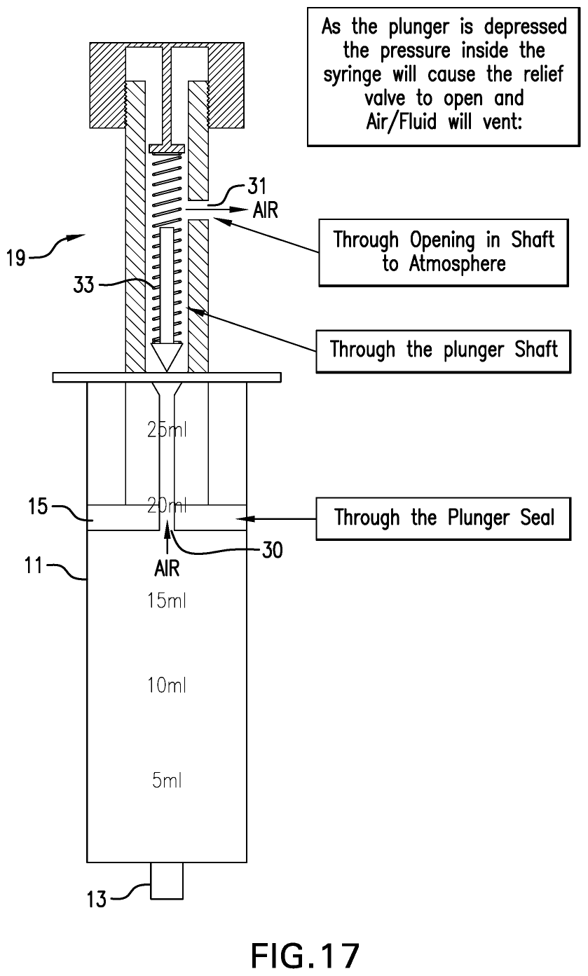

FIGS. 16-18 illustrate an example, and use, of a pressure relief valve system incorporated into a screw-type syringe plunger 19. As shown, the plunger 19 will have an opening 30 in its plunger seal 15 and an outlet opening 31 at another point in the plunger 19. As seen in FIG. 17, air enters opening 30 as the plunger seal 15 is forced downward and air exits outlet opening 31 after passing through the plunger shaft channel 33. Such a pressure relief system may be added anywhere in the system between, and including, the syringe plunger seal 15 and the balloon 16. This valve may need a closed position for the volumetric fill part of the method. As the screw plunger is tightened, the pressure required to open the valve would increase. This system could be preset or a variable system controlled by the clinician. Any of the variable systems discussed herein could include a scale or display of the pressure setting.

In yet another embodiment, a band may be utilized to prevent stretching. Bands are typically made of pliable material, (e.g. vinyl) and easy to manufacture, can be made clear, are easy to attach additional part to by welding or adhesives, and are safe and generally comfortable for the patient. Additional parts may be balloon or Velcro®, for example. Once the syringe is disengaged, the bands tend to relax, and exerted force asymptotically as it approaches a limit. This is due to the balloon changing shapes and the band stretching or relaxing. For a snug or correctly applied band, the force when all of the air is removed at the end of the procedure is less than when the band is first applied. At this point, both balloons are at 0 ml inflation, but the final force (in lbs) is lower than the initial force (in lbs), and the force (in lbs) at the end increases slightly over time. This is due to the stretching of the band during the procedure.

The band may be constructed of a material that limits stretching when the band is applied or during use. Additionally, the balloon or bladder may be constructed in such a way as to prevent backpressure associated with balloon inflation. Examples include, but are not limited to, an oversized balloon or a balloon made from a limited stretch material. Reinforcement materials could be added to the bands to help prevent stretching, or the bands could be made of materials that prevent stretching.

Another method of application to create uniform compression force with a bypass-type plunger system of the present invention would be to slightly inflate the balloon prior to applying the band to the patient. The balloon may be inflated with the syringe, supplied by the manufacturer inflated, or the balloon may be formed around a soft foam to keep it open prior to being applied to the patient. The band would be applied to the patient in a way that would compress the pre-inflated balloon. The syringe would have an opening ending at the desired inflation volume. The syringe plunger would be pulled back beyond the volumetric inflation point, and then, when inserted into the check valve, the balloon would be allowed to relax. As the plunger is depressed below the opening, it would deliver a desired volume of air to the relaxed balloon. This would increase the consistency of the compression force from patient to patient.

In yet another embodiment, a plunger may be modified to measure the air pressure delivered by the syringe into the inflatable band. The syringe plunger may utilize a spring allowing the plunger to move and a feedback mechanism indicates to the user the air pressure supplied by the syringe. The diameter of the plunger seal in conjunction with the force pushing on the plunger by the user determines the pressure of the fluid delivered. If one pound of force is applied to a one square inch plunger seal, then one psi will be delivered. Changing of the force pushing on the plunger would have proportional changes to pressure, while changes to the surface area of the plunger seal would have inverse affects on the pressure delivered.

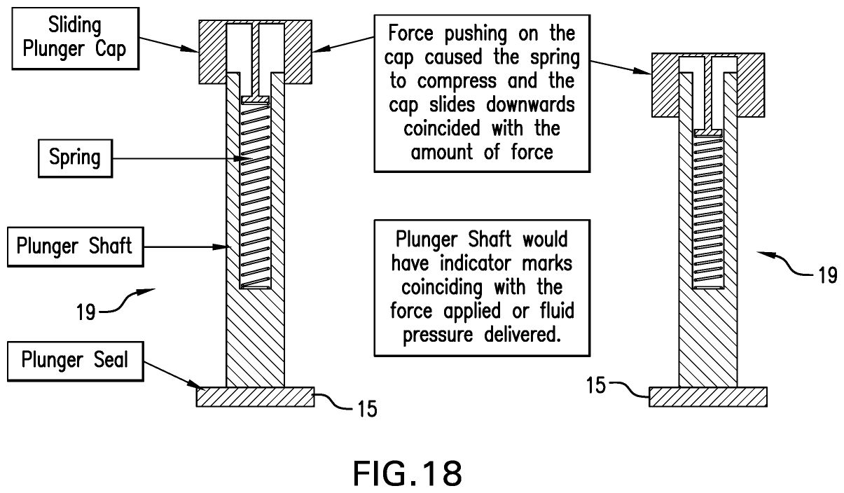

FIG. 18 is a sketch of the compressible feedback syringe plunger (without the syringe barrel). FIG. 18 shows force being applied to the top of the plunger indicated by the plunger cap and the spring allows the cap to slide downwards coinciding with the amount of force applied to the cap. The plunger seal, inside the syringe barrel, then translates that force to a deliverable fluid pressure.

The end of the plunger may slide over the bottom portion of the plunger or into the bottom portion of the plunger. The indicator marks may be on the top or bottom portion of the plunger. The marks on the plunger may be to indicate force pushing on the cap or deliverable fluid pressure. The marks may be a color scheme based on usage of the product. The sketch above is only mean to indicate the idea and not a finished device.

### EXAMPLES

### Example/Experiment 1: Test Apparatus

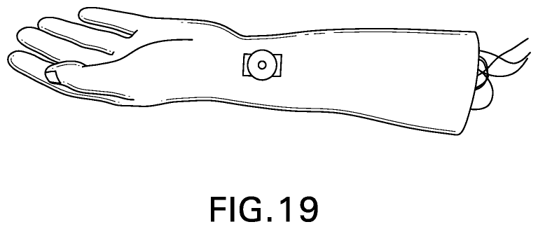

To create a test model to measure compression (shown in FIG. 19), a load cell was inserted into the wrist of a silicone arm model over the radial artery position. The load cell was connected to an excitation power and to a data acquisition system. The load cell was calibrated using a force gauge. The purpose of this model was to measure compression over the radial artery. For example, this compression could be manual, a clamp system, or a circumferential inflatable band. For compression studies, the data acquisition was set to pull data every second, and converted to minutes for the graph. The data was measured in lbs, and can be converted to pounds per square inch (psi) by dividing the lbs over the surface area to which it is applied. Pressure can be converted from psi to millimeters of Mercury (mmHg) (to relate to blood pressure measurements) by multiplying the psi by 51.7 (mmHg/psi).

In the particular test model shown in FIG. 19, a 30 mm×18 mm (3 mm thick) plastic square is fitted over the load cell to simulate a StatSeal RAD Disc® available from Biolife.

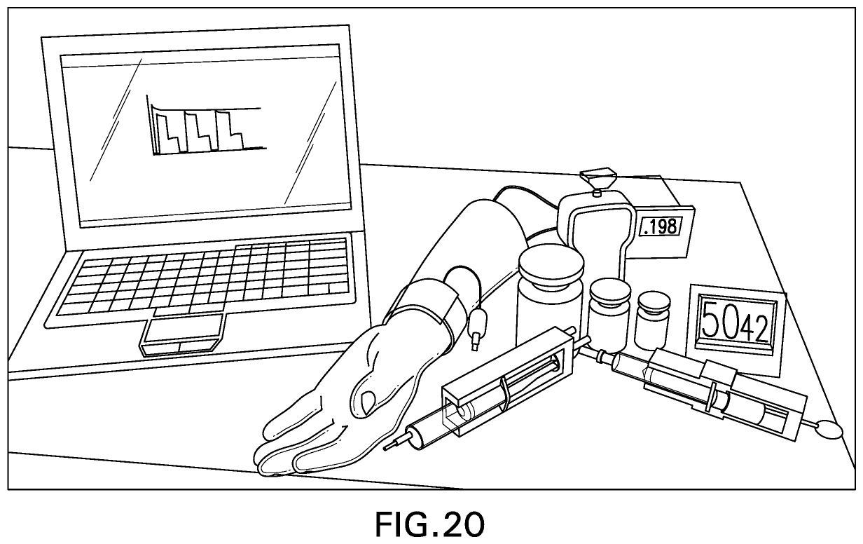

FIG. 20 shows more of the test model system with the data acquisition system connected. Note the TR Band® around the wrist model over the load cell position.

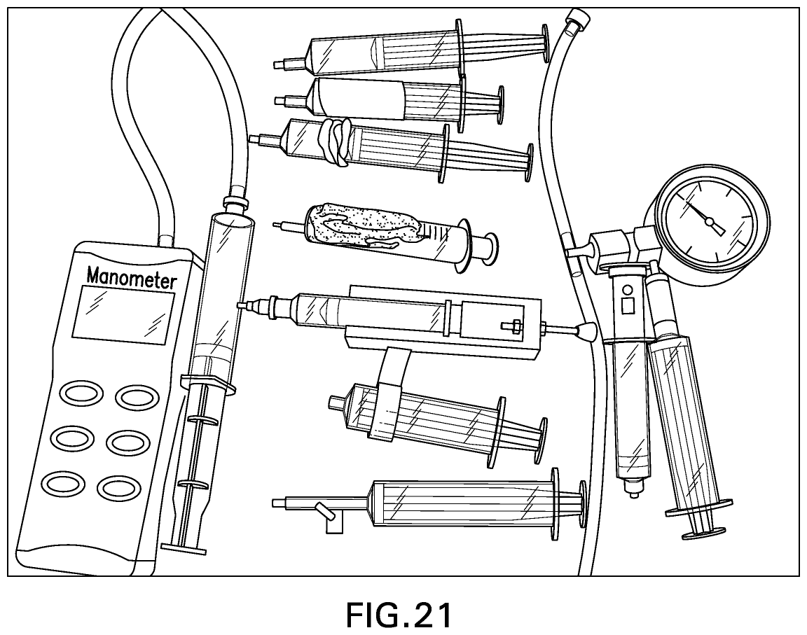

FIG. 21 illustrates different syringes and setups used for testing a particular patient.

### Example/Experiment 2: Simulated Actual Procedure

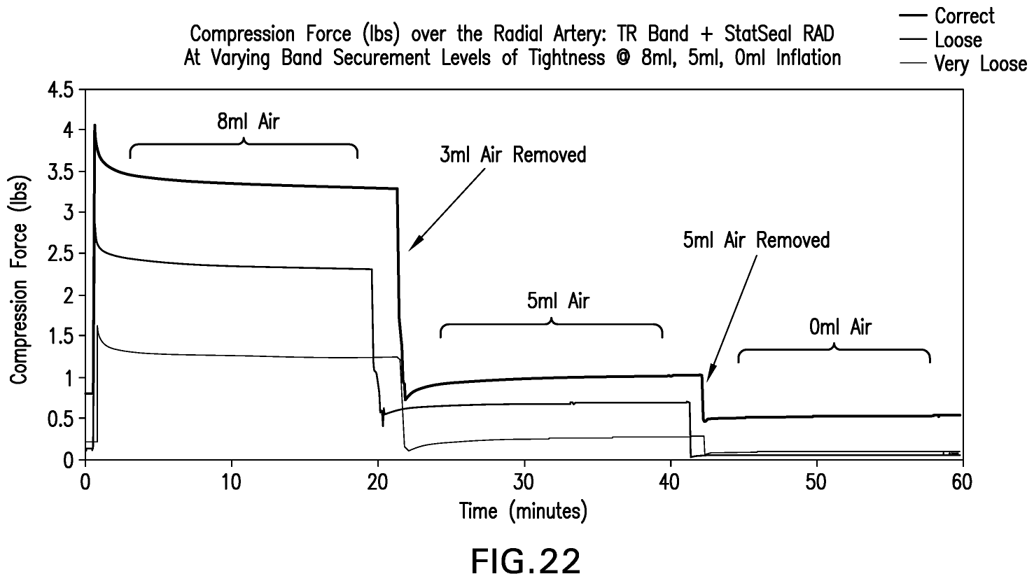

The graph in FIG. 22 shows the compression for exerted over the radial artery for a typical actual use, or a TR Band® and a StatSeal RAD Disc®. After the procedure, the disc is placed over the insertion site. It is large enough to cover the wounds in the skin and artery. To prevent dislodgement, the band was secured in place by wrapping it around the patient, such that the center point indicator of the balloon is centered over the disc. The balloon was inflated with 8 ml of air (the inflation should be performed in less than 5 seconds). The sheath was pulled from the artery while the balloon is being inflated.

The balloon was left in place with 8 ml of air for approximately 20 minutes. At that point, 3 ml of air was pulled from the balloon, leaving the balloon with 5 ml of air remaining. This is only approximate, due to pressure differentials of injected and removed air volumes, and due to the non-linear relationship the Ballou's material has between its internal pressure and volume. The 5 ml-inflated balloon was left on the patient for approximately 20 more minutes, after which the remainder of the air was removed. The fully deflated band remained on the patient for another 20 minutes. If, at any point during the procedure, bleeding or a hematoma occurs, additional air was injected back into the balloon to increase compression to control the bleeding or hematoma.

As seen in the graph of FIG. 22, varying the securement tightness upon application of the band has impact on the compression applied as the balloon is inflated with the same volumes of air. The bands can also be applied too tightly, but this is far less common than applying the bands too loosely. The graph of a band applied too tightly would be above that of the correctly applied band, indicating that applied force at all levels of inflation are higher for a band applied too tightly than for a properly applied band.

The graph of FIG. 22 represents a normal compression scenario for hemostasis using a TR Band® and a StatSeal RAD® Disc.

### Example/Experiment 3: Determining Band Securement Levels of Tightness

Defining and differentiating between snug/correct, loose and very loose band securements may prove challenging. The terms snug/correct, loose, or very loose are not exact measurements and are relative for the set of tests being performed at the time. Variations in securement associated with these terms will impact the lbs during the inflation steps. These tests are all on the same model; the securement issues compound when the patient and clinician involved are included as variables in the analysis.

Bracelets for the bands are of different lengths and wrists of patients come in different sizes, so there is no easy way to create a measurement system for a clinician to use to set a band at the correct tightness.

For these experiments, the wrist model had a circumference of 21 cm. The TR Band® had a length of 24 cm. Overlap of the Velcro® securement was measured to determine the level of securement. If the overlap was greater than 2.3 cm, it was determined to be too tight. An overlap of 2.0-2.3 cm was determined to be the correct amount of securement in this particular example. An overlap of 1.5-2.0 cm was loose and an overlap of less than 1.5 cm of overlap was very loose.

A measurement system for band securement would not translate to actual usage. Expecting clinicians to measure each patient's wrist, calculate the amount of overlap required, and then measure that amount of overlap is not practical in the real world due to time constraints and because blood smudges would obscure reading on measurement devices.

### Example/Experiment 4: Hole in Syringe

A 1/16″ hole was drilled into the barrel of the syringe at the 8 ml mark. When air is being injected and the plunger seal is above the 8 ml mark, the air will be allowed to escape from either the outlet of the syringe or through the hole in the barrel. Where the air leaves the syringe will depend on several variables, including the cross-sectional ratio between the outlet hole and the side hole, the speed at which the plunger is being pushed, and the external pressures that are being exerted on the outlet hole and the side hole. The outlet hole in the syringe will be connected to the balloon. Therefore, the external pressures (i.e. how tightly the band is applied, the geometry of the wrist, etc.) will impact the external pressure on the outlet of the syringe.

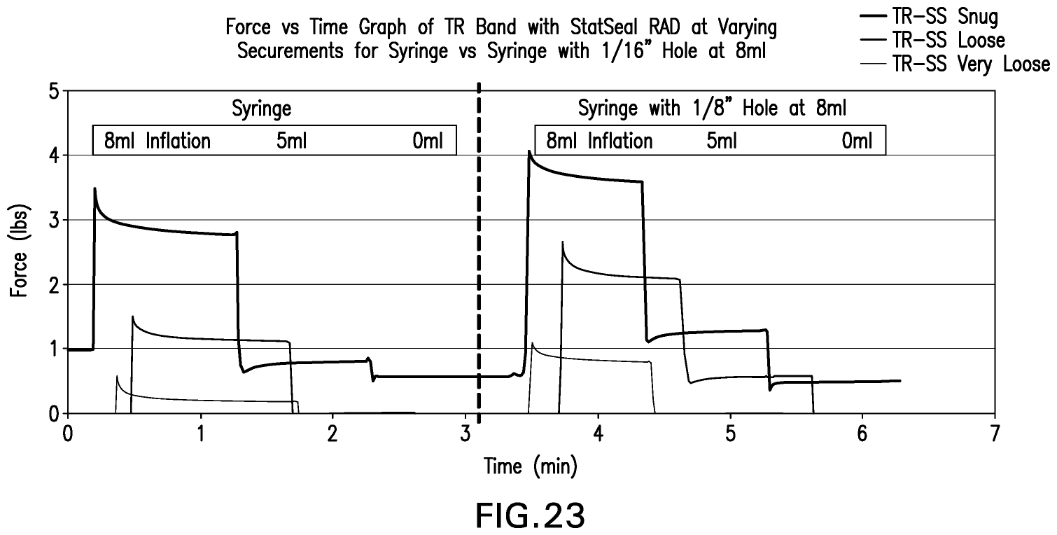

The graph in FIG. 23 demonstrates the advantage that the syringe with an 1/16″ hole at 8 ml has over an unmodified syringe. The leftmost peak corresponds with the injection of the TR Band® with StatSeal RAD® (TR-SS) with 8 ml of air from an unmodified syringe. The first steep decline corresponds with the removal of 3 ml of air from the band, and the second steep decline corresponds with the removal of the remaining air from the band. Although the second steep decline cannot be seen in the TR-SS Loose or TR-SS Very Loose data series, the associated deflations are nonetheless present. The rightmost peak corresponds with the injection of air from the syringe with an 1/16″ hole at 8 ml, performed by drawing the plunger to the 20 ml mark and plunging the contents of the syringe into the band. Similarly to the procedure regarding the unmodified syringe, the first steep decline corresponds with the removal of 3 ml of air from the band, and the second steep decline corresponds with the removal of the remaining air. Although the second step decline cannot be seen in the TR-SS Very Loose data series, the associated deflation is nonetheless present.

Note there is not a significant impact in force (lbs) from the type of syringe used for a snug or correctly applied band, but there are significant increases in force when using a loose or very loose band. With the snug band, the 8 ml initial inflation created approximately 33% increase in force, while with the loose bands, the 8 ml initial inflation created approximately 100% increase in force. This test demonstrates that a hole in the syringe assists in overcoming band securement issues by partially filling the initial deflated band with a volume of air. The hole at 8 ml also allows the syringe to inject 8 ml volume of air into the balloon after it has been inflated to atmospheric pressure. The location and size of this hole could vary and would create different results allowing for different uses beyond catheter insertions.

FIG. 23 illustrates the difference in compression force created with a syringe and that created with a syringe having a 1/16″ hole in the barrel at 8 ml at varying securements of the band.

### Example/Experiment 5: Balloon on Table

A TR Band® was placed directly onto a table and inflated using two methods. The first method used inflated the band using the syringe with a ⅛″ hole at the 8 ml mark on the syringe, while the second method inflated the band using an unmodified syringe before relaxing the band with the syringe with a ⅛″ hole. This study was performed to compare the effectiveness of the two methods at pressurizing the band prior to volumetric injection.

The first method was performed by placing the band directly on the table and inflating it with the syringe with a ⅛″ hole. The plunger of the syringe with a ⅛″ hole was drawn to 20 ml, the nozzle of the syringe was inserted into the band, and the syringe was depressed slowly until the seal of the plunger was approximately 2 ml above the hole (that is, the plunger was approximately at 10 ml). The syringe was then removed from the band. The band was allowed to relax, and the volume of air within the band was then measured by removing all of the air with an unmodified syringe.

The second method was performed by placing the band directly on the table, inflating it with an unmodified syringe, and venting it using the syringe with a ⅛″ hole. The plunger of the unmodified syringe was drawn to 6 ml, the nozzle of the syringe was inserted into the band, and the syringe was fully depressed. The unmodified syringe was removed. The plunger of the syringe with a ⅛″ hole was drawn above the hole (that is, drawn above 8 ml), and the nozzle of the syringe with the hole was inserted into the band. The band vented until it was in equilibrium with atmospheric pressure, the syringe was removed, and the band was allowed to relax. The volume of air within the band was measured using an unmodified syringe.

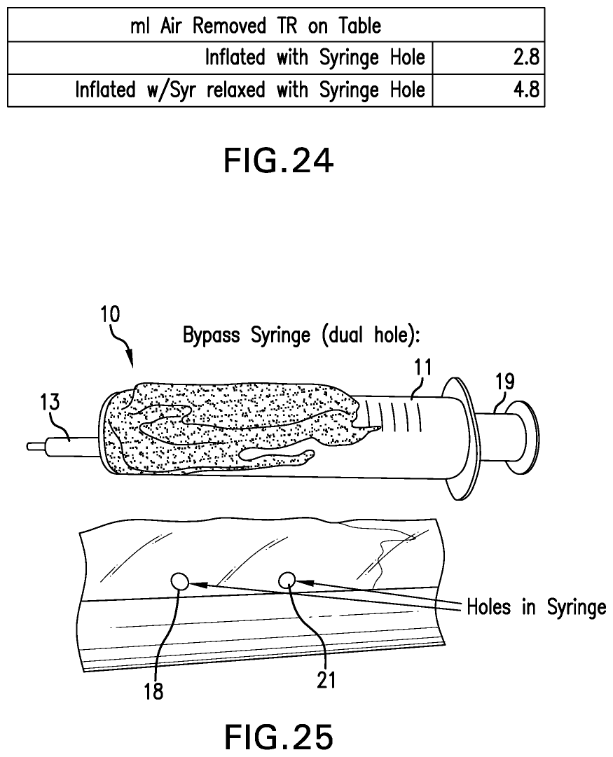

The results of the study are displayed in the table in FIG. 24. It was demonstrated that the second method resulted in significantly more air remaining in a relaxed band if air is forced into the balloon via the second method versus a passive inflation using the first method. This makes clear the advantages of a pressurized pre-inflation step with an unmodified or unmodified-simulating syringe prior to a relaxation step. This testing shows the use of a pre-inflation step for priming the band for hemostasis to further overcome variable of band securement. Pre-inflation results in as much as 4.8 ml of air remaining in a relaxed band versus 2.8 ml if a syringe with a hole is used to pre-inflate. The pressurized pre-inflation balloon contained a 71.4% increase in entrained air from the relaxed balloons.

The table of FIG. 24 shows the difference in entrained air from a relaxed balloon band resting on a table after inflating it with the syringe having a ⅛″ hole and after inflating it with an unmodified syringe and relaxing using the syringe having a ⅛″ hole.

### Example/Experiment 6: Bypasses

An alternative embodiment of a syringe was created that would force over 5 ml of air into a balloon, and then allow the balloon to relax based on how tightly the band was secured on the model or patient. The syringe was modified in such a way as to pressurize the balloon, allow air to bypass the plunger and vent to atmosphere, and finally to deliver a volume of air into the balloon.

To create this modified syringe shown in FIG. 25, two ⅛″ holes were drilled into the barrel of a TR Band® syringe, at 8 ml and 15 ml. A small plastic channel was epoxied to the outside of the barrel of the syringe creating an airtight channel over the two holes through which air or another fluid could pass. When the plunger seal is above both holes, an airtight system is created and the syringe will displace fluid as the plunger is depressed. When the plunger seal is between the holes, air can vent around the seal through the holes and the channel to into atmosphere. Once the plunger is below the bottom hole, the syringe will displace a volume of air/fluid depending on the location of the bottom hole.

Another alternative bypass syringe was created, as shown in FIG. 26, by creating a slit in the side of the syringe and covering the slit with an adhesive tape (such as duct tape). This created a bypass channel in the barrel of the syringe that could be easily manufactured using an adhesive or a shrink wrap around the syringe barrel.

The bypass channel syringe of the present invention has advantages over the syringe with a 1/16″ hole alone when the band is very loosely secured. In a study comparing a bypass syringe to an unmodified syringe and according to the procedure described in “Example/Experiment 4: Hole in Syringe,” using a very loose band, resulted in a peak force of approximately 2 lbs, nearly identical to the peak force corresponding with use of a loose band and twice the peak force in the very loose trial of the study analyzing the syringe with a 1/16″ hole. This comparison indicates that the risk of hematoma or bleeding resulting from a band being applied too loosely can be almost entirely eliminated by using an iteration of the bypass syringe.

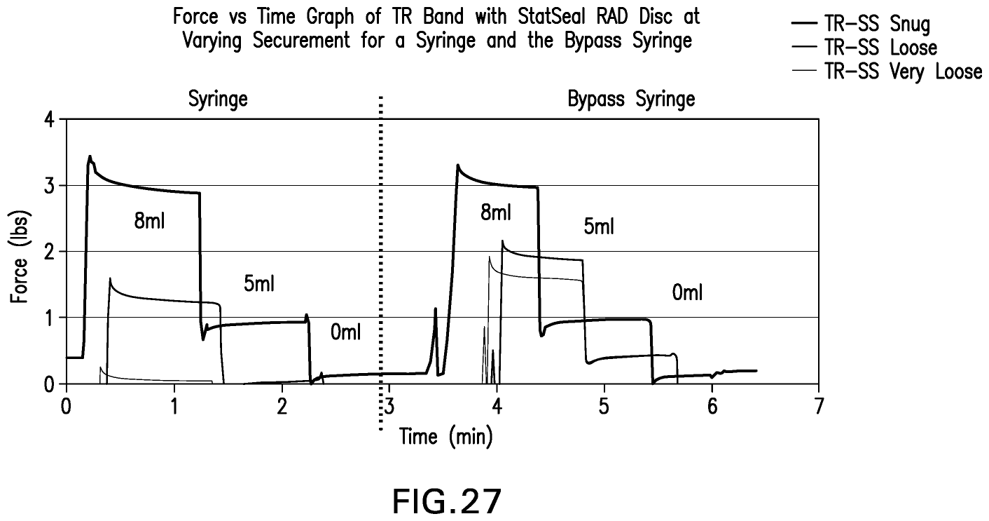

The graph in FIG. 27 illustrates the difference in of compression force created with a syringe versus a bypass syringe at varying securements of the band.

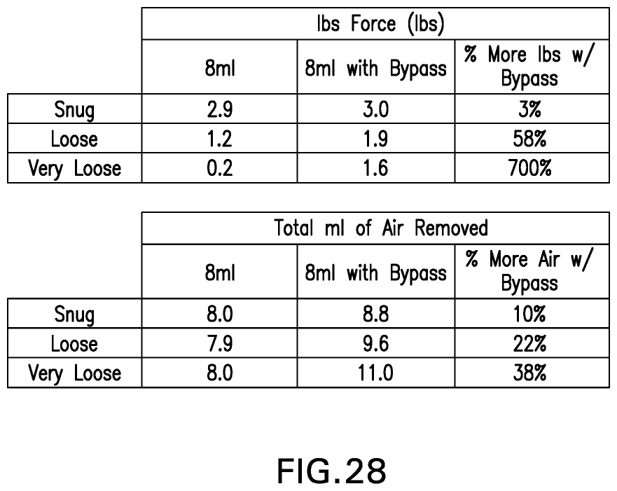

The table in FIG. 28 illustrates the compression force (lbs) and volume (ml) of air removed from the fully inflated balloon bands in FIG. 26.

In the experiment above, the “Very Loose” band was so loose on the model that 8 ml of air inflated from a regular syringe did not apply a compression force over the load cell in the model, but the 11 ml of air that was injected from the 8 ml bypass syringe was enough air to create 1.6 lbs of compression force over the load cell. When the band was applied snugly, the initial compression force against the balloon, resulting from being tight against the model, prevented an excessive amount of air from being injected into the balloon as only 8.8 ml of air was removed.

### Example/Experiment 7: Pressure Relief Valve

A pressure relief valve was inserted into the syringe such that it would vent to the atmosphere if the internal pressure of the band became too strong. The pressure relief valve could be connected anywhere on the syringe, nozzle, tubing, check valve or balloon.

To test how the relief valve would perform for bands of different levels of securement, and how resilient the valve is to injection speeds, a syringe with a pressure relief valve was attached to the nozzle of a syringe and TR Band® nozzle was attached to the other side of the pressure relief valve. The first part of the experiment was run by pulling the plunger of the syringe to 20 ml and injecting the air directly into a snug band and into a loose band at three injection speeds. As demonstrated, both the snug and loose bands created similar compression forces (lbs) over the load cells and in all tests the pressure relief valve vented to the atmosphere. Between inflations, the band was completely deflated and allowed to rest. Injections were performed at slow, normal, and fast injection speeds, corresponding with the left, middle, and right peaks of the graph, respectively. This study demonstrated that, by implementing a pressure relief valve in the nozzle of the syringe, both the securement of the band and the speed of injection were overcome as process variables of inflation. The forces (lbs) over the load cell are higher than in other tests, but this could be corrected by using the correctly calibrated or designed pressure relief valve. Additionally, a variable pressure relief valve could have been employed.

The snug and loose bands were able to create similar pressures because more air was inserted into the loose band than into the snug band. The snug band averaged 13.4 ml of inflation, while the loose band averaged 16.4 ml over the 3 inflations of each band securement. The loose band was inflated with 20.6% more air than the snug band to create a similar force over the load cells. Note that the loose bands created slightly less force (lbs) with the pressure inflation than did the snug bands.

The second iteration of the study used a loose band only and included an extreme injection speed. On the extreme injection trial, the data became inconsistent with previous trials, as the band failed to inflate properly. This happened because the extreme injection overwhelmed the orifice in the syringe nozzle, creating a pressure in the barrel of the syringe that triggered a premature release of fluid by the relief valve. This resulted in too little air being injected into the balloon, creating too little force, as demonstrated in the graph, over the load cell. This demonstrates that the real and the perceived speeds of inflation can be dependent on the technician inflating the band. However, it is not expected that this will be an issue for the pressure relief valve syringe because the speed of injection was above any that would be expected in actual use on a patient. Secondly, even if such an extreme speed inflation were performed, the failure of the pressure relief valve would prevent over inflation of the balloon and would pose no risk of injury to over compression to the patient.

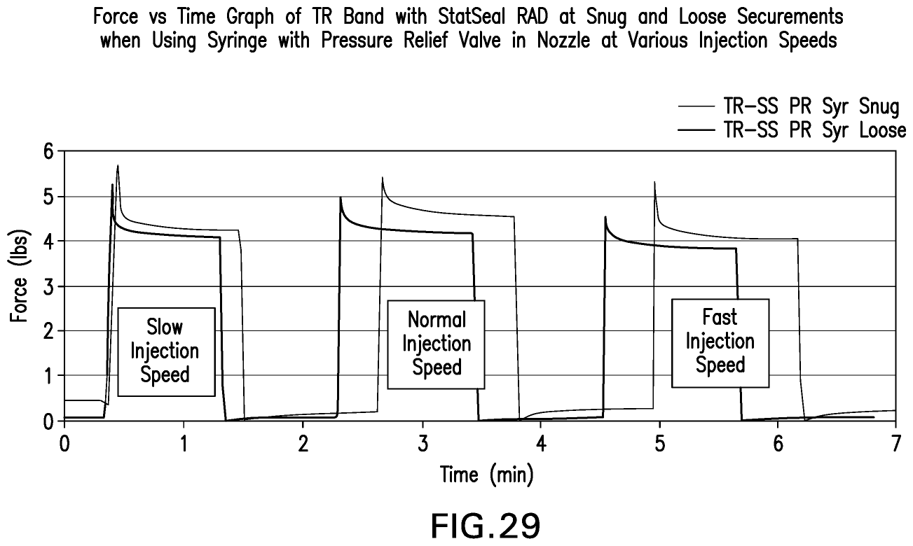

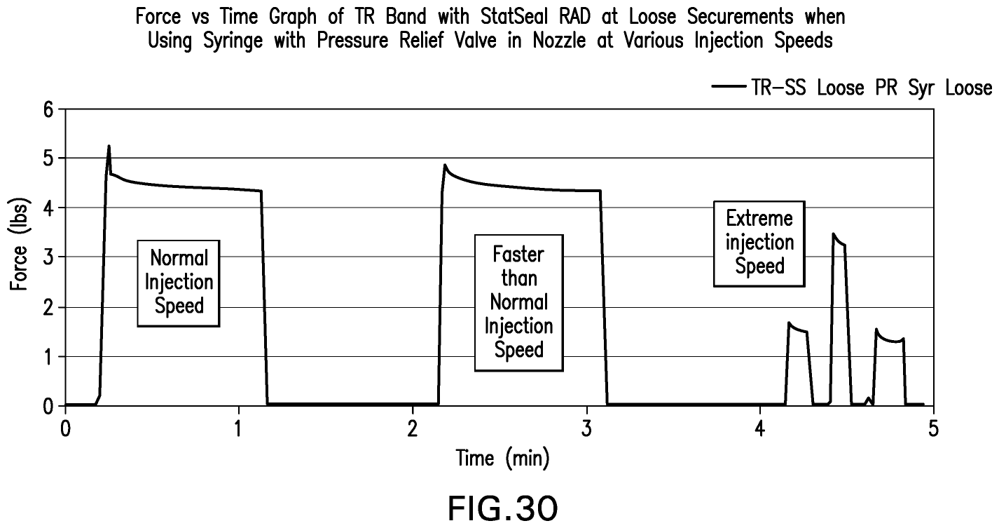

The graphs in FIGS. 29 and 30 represent the force applied by the TR Band® with a StatSeal RAD at various speeds of injection. Note the breakpoint of the data at the very fast injection speed.

### Example/Experiment 8: Pressure Inside the Balloon

A syringe was attached to a pressure gauge manometer and the TR Band® with a StatSeal RAD. The balloons were inflated to a pressure reading on the manometer, and the syringe was quickly disconnected from the check valve. The purpose of this test was to demonstrate the impact of internal pressure versus compression force (lbs) over the load cell.

The band was inflated until the manometer read the desired pressure, at which point, the connection to the band was severed. The band was allowed to relax for approximately 1 minute, then it was deflated completely for 1 minute. This procedure was performed such that the balloon was inflated to an initial internal pressure of 1 psi, 2 psi, 3 psi, 4 psi, and 5 psi. These iterations are represented as peaks on the graph, with an internal pressure of 1 psi corresponding to the leftmost peaks and an internal pressure of 5 psi corresponding to the rightmost peaks. It was found that an internal pressure of between 2 psi and 3 psi may be used for patent hemostasis, but other pressures are also acceptable. This study also demonstrates that, when the band is inflated to a given pressure rather than a given volume, much of the dependence on securement for force exertion of the band is removed. Note again that the loose band created less compression force (lbs) over the load cell.

Note in the graphs the continual reduction in compress over the load cell for each test. This is not due to air leaking from the balloon/valve, but from the balloon and band stretching and relaxing. This would also have impacted the internal air pressure of the balloon, and it why the test was only run to inflate the balloon to an initial pressure to simulate clinical usage.

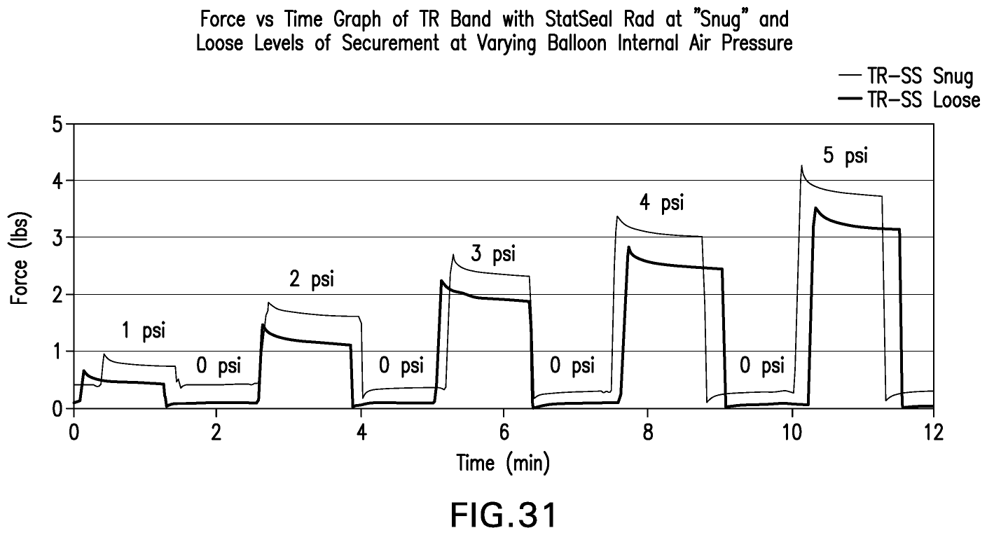

The graph in FIG. 31 illustrates the correlation of internal pressure to external force applied by TR Band® with StatSeal RAD. The leftmost peak correlates with 1 psi, and each subsequent peak has a 1 psi increase in internal pressure. Note how little difference there is between snug and loose securements of the band.

### Example/Experiment 9: Comparative Assessment

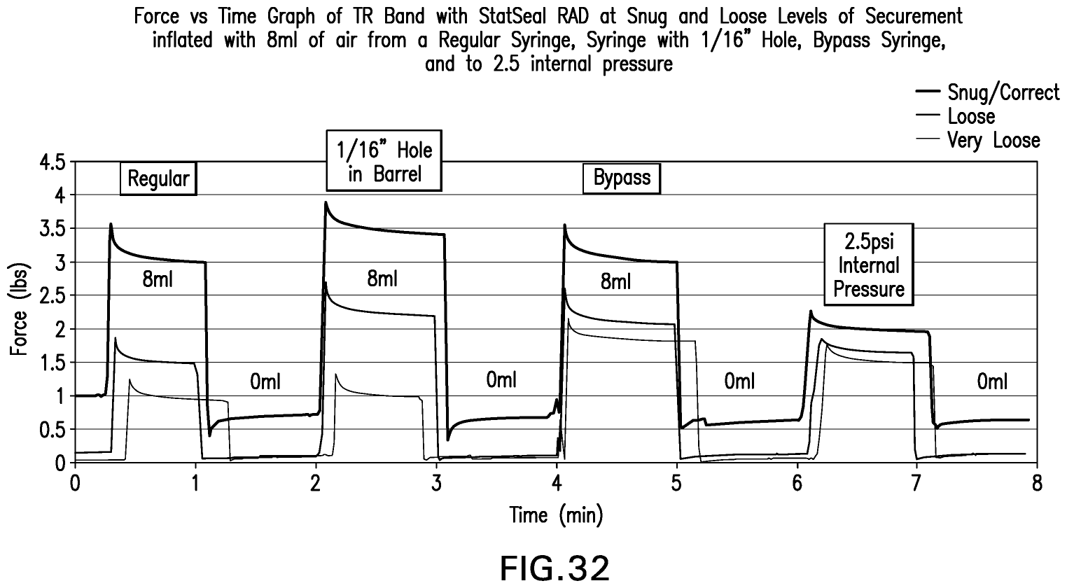

The TR Band® with StatSeal RAD beneath was attached to the arm model snuggly/correctly and inflated with 8 ml of air from a regular TR Band® syringe for 1 minute hence all of the air was removed from the balloon. As shown in FIG. 32, a syringe with a 1/16″ hole at the 8 ml mark was used to inflate the balloon for 1 minute. Afterwards, it was deflated for 1 minute. Next, a bypass syringe was employed and then, finally, the balloon was inflated to 2.5 psi.

The modified syringes generally outperformed the regular syringe, with the most consistent performer being the internal pressure syringe. Even that inflation method was impacted by band securement tightness, with the correctly secured band creating ˜33% more lbs of force over the radial artery than the loosely fit band. Even the pressure-controlled balloon band may need additional manipulation, thus the need for the non-pressure-controlled portion in the bottom of the barrel of the syringe.

Again, the bypass syringe created a force profile for all securement levels of band that would work in a clinical setting with one partial deflation step followed by a full deflation. But like the pressure-controlled syringe, it would need a bottom portion of the syringe to manipulate the balloon.

What is claimed is:

1. An inflation device comprising a syringe barrel, a syringe plunger shaft, a syringe nozzle, and a syringe plunger seal wherein the syringe barrel comprises a fluid bypass channel beginning at a first position on the syringe barrel and ending at a second position on the syringe barrel wherein the distance between the first position and the second position is greater than the width of the syringe plunger seal such that when syringe plunger shaft is pushed toward the syringe nozzle, the syringe plunger seal may be positioned between the first position and the second position so that fluid may flow around the syringe plunger seal to an area behind the syringe plunger seal.

2. The inflation device of claim 1 wherein the syringe barrel comprises a continuous bypass channel connected to the syringe barrel at two channel openings.

3. The inflation device of claim 2 wherein one channel opening is located at a position on the syringe barrel beginning at a point corresponding to 10 to 65% of the total volume of the syringe barrel and one channel opening is located at a position on the syringe barrel at a point corresponding to 25 to 80% of the total volume of the syringe barrel.

4. The inflation device of claim 2 wherein the continuous bypass channel is formed integrally with the syringe.

5. The inflation device of claim 1 comprising multiple bypass channels.

6. The inflation device of claim 5 wherein none of the multiple bypass channels are in fluid contact with another of the multiple bypass channels.

7. The inflation device of claim 5 wherein at least one of the multiple bypass channels is in fluid contact with another of the multiple bypass channels.

8. The inflation device of claim 1 comprising multiple continuous bypass channels.

9. The inflation device of claim 2 further comprising a structure for forming an airtight seal over the channel openings to create a bypass channel.

10. The inflation device of claim 9 wherein the structure is chosen from the group consisting of a circumferential overwrap material for placing over the syringe barrel and covering the channel openings, a material that may be removably adhered over the channel openings, and a material permanently adhered over the channel openings.

11. An inflation device comprising a syringe barrel, a syringe plunger shaft, a syringe nozzle, and a syringe plunger seal wherein the syringe barrel comprises a fluid pressure structure that vents fluid to the atmosphere outside of the syringe barrel, wherein the fluid pressure relief structure comprises a hole in the syringe barrel and wherein the hole in the syringe barrel
a. has a minimum diameter of 1/32″ for syringes having volumes of 5 ml or less;
b. has a maximum diameter of 15% of the diameter of the syringe plunger seal for syringes having volumes larger than 5 ml; and
c. is located at a position on the syringe barrel beginning at a point corresponding to 10 to 65% of the total volume of the syringe barrel.

12. The inflation device of claim 11 wherein the syringe barrel comprises multiple holes.

13. An inflation device comprising a syringe barrel, a syringe plunger shaft, a syringe nozzle, and a syringe plunger seal wherein the syringe barrel comprises pressure relief valve located at a position on the syringe barrel beginning at a point corresponding to 10 to 65% of the total volume of the syringe barrel.

14. The inflation device of claim 13 wherein the pressure relief valve is a fixed vent pressure valve.

15. The inflation device of claim 13 wherein the pressure relief valve is a variable vent pressure valve that may be set to a desired pressure relief.

16. The inflation device of claim 15 wherein the variable vent pressure valve comprises a pressure indicator.

17. An inflation device comprising a syringe barrel, a syringe plunger shaft, a syringe nozzle, a syringe plunger seal, and a pressure relief valve, wherein the pressure relief valve is in fluid contact with the syringe nozzle.

18. The inflation device of claim 17 wherein the pressure relief valve is a fixed vent pressure valve.

19. The inflation device of claim 17 wherein the pressure relief valve is a variable vent pressure valve that may be set to a desired pressure relief.

20. An inflation device comprising a syringe barrel, a syringe plunger shaft, a syringe nozzle, and a syringe plunger seal comprising a through-hole, wherein the syringe plunger shaft is hollow and comprises a pressure relief valve system for venting fluid through the shaft.

21. The inflation device of claim 20 wherein the pressure relief valve system comprises a compression mechanism, a spring, and a seal face.

22. The inflation device of claim 20 wherein the pressure relief valve system comprises a fixed vent pressure valve.

23. The inflation device of claim 20 wherein the pressure relief valve system comprises a variable vent pressure valve that may be set to a desired pressure relief.

24. The inflation device of claim 23 wherein the variable vent pressure valve system comprises a pressure indicator.

25. The inflation device of claim 24 wherein the variable vent pressure valve system comprises structure for adjusting the vent pressure from the syringe plunger shaft.

26. The inflation device of claim 21 wherein the compression mechanism comprises a screw tightening mechanism which, when tightened, further compresses the spring, resulting in a greater internal pressure of the syringe barrel for dislodgement of the seal face.

27. An inflation device comprising a syringe barrel, a syringe plunger shaft, a syringe nozzle, and a syringe plunger seal, wherein the syringe barrel comprises a pressure relief valve system 10-30% from the bottom of the barrel.

28. The inflation device of claim 27 wherein the pressure relief valve system comprises a fixed vent pressure valve.

29. The inflation device of claim 27 wherein the pressure relief valve system comprises a variable vent pressure valve that may be set to a desired pressure relief

30. A compression device comprising a mechanism for relieving pressure, a circumferential band, and housing, the housing comprising an expandable bladder for applying pressure to a skin wound and a vascular wound, the bladder being partially inflated prior to use on the patient.

31. The compression device of claim 30 wherein said expandable bladder may be delivered to the user partially inflated or may be inflated by the user prior to use.

32. The compression device of claim 30 wherein said expandable bladder contains a foam or other material intended to simulate a pre-inflated condition.

33. A compression device comprising a circumferential band and housing, the housing comprising an expandable bladder for applying pressure to a skin wound and a vascular wound and the circumferential band comprising a limited-stretch material.

34. The compression device of claim 33 wherein the expandable bladder is engineered to expand to create sufficient hemostasis compression without such that the internal pressure of said expandable bladder is transferred directly onto the wound.

35. The compression device of claim 33 wherein the expandable bladder comprises a non-stretching balloon or circumferential wrap.

36. A method of applying a compression device to a patient, wherein the compression device comprises a wrapping band and an inflatable balloon for creating compression against the patient and wherein the method employs a balloon that is partially pre-inflated to overcome band securement variabilities when applying and securing the band to a patient comprising the steps of:
a. partially inflating the balloon connected to the band with an amount of fluid that will inflate the balloon to below the point of causing the materials from which the band is constructed to stretch;
b. applying the band to the patient by circumferentially wrapping the band around a body part of the patient before or after partially inflating the balloon;
c. releasing an amount of air from the balloon corresponding to the volume of air created in the balloon due to the increased air pressure in the balloon caused by securing the band to the patient's body part;
d. inflating the balloon with a volumetric dose of air to create more consistent force over the artery than can be achieved without the pre-inflation step.
